(19) United States
(12) Patent Application Publication
(10) Pub. No.: US 2009/0298792 A1
(43) Pub. Date: Dec. 3, 2009

(54) Anti-Inflammatory Polymer

(75) Inventors: Andreas Grassauer, Vienna (AT); Christiane Meier, Seyring (AT)

Correspondence Address: FULBRIGHT & JAWORSKI L.L.P., 600 CONGRESS AVE., SUITE 2400, AUSTIN, TX, 78701, US

(73) Assignee: MARINOMED BIOTECHNOLOGIE GMBH, Vienna (AT)

(21) Appl. No.: 12/296,022

(22) PCT Filed: Mar. 30, 2007
(86) PCT No.: PCT/EP07/02864

§ 371 Date: Oct. 3, 2008

(30) Foreign Application Priority Data
Apr. 4, 2006 (EP) 06450051.5
Apr. 4, 2006 (EP) 06450052.3

(51) Int. Cl. A61K 31/722; A61K 31/717; A61P 37/02

(52) U.S. Cl. 514/55; 514/57

(57) ABSTRACT

The present invention provides the use of cellulose sulfate or chitosan for the manufacture of an anti-inflammatory pharmaceutical or cosmetic composition for the treatment of inflammation.

The present invention relates to the field of immunology and anti-inflammatory agents.

Anti-inflammatory medicaments can be classified into those of steroid and of nonsteroidal type. Steroid anti-inflammatory compounds are still the most effective ones in the treatment of inflammatory diseases and conditions such as: asthma, chronic obstructive pulmonary disease, inflammatory nasal diseases such as allergic rhinitis, nasal polyps, intestinal diseases such as Crohn's disease, colitis, ulcerative colitis, dermatological inflammations such as eczema, psoriasis, allergic dermatitis, neurodermatitis, pruritis, conjunctivitis and rheumatoid arthritis. In addition to excellent potency and effectiveness, medicaments of this type also possess numerous unfavourable side-effects, (e.g. disturbance of carbohydrate metabolism, decreased calcium resorption, decreased excretion of endogenous corticosteroids and disturbance of physiological functions of the pituitary gland, adrenal cortex and thymus.

Psoriasis is a common skin disease characterized by hyperplasia of keratinocytes resulting in thickening of the epidermis and the presence of red scaly plaques. The lesions in this chronic disease typically are subject to remissions and exacerbations. There are several patterns, of which plaque psoriasis is the most common. Guttate psoriasis, with raindrop shaped lesions scattered on the trunk and limbs, is the most frequent form in children, while pustular psoriasis is usually localized to the palms and soles. The classical inflammatory lesions vary from discrete erythematous papules and plaques covered with silvery scales, to scaly itching patches that bleed when the scales are removed. Despite a voluminous scientific literature and numerous treatment strategies, there is still no effective treatment for psoriasis that is completely without side effects.

The number of different and sometimes toxic treatments employed for amelioration of psoriasis is testimony to the resistant nature of this disease. The devastating nature of this disease is emphasized by the extent of the side effects that psoriasis sufferers are willing to endure to attain a remission to a disease that they know will recur sooner or later.

It is a goal of the present invention to provide anti-inflammatory agents for the treatment of various diseases, especially skin diseases including psoriasis, related to immunoreactivities.

The present invention provides the use of a polymer for the manufacture of an anti-inflammatory pharmaceutical or cosmetic composition for the treatment of inflammation, wherein the polymer is cellulose sulfate or chitosan. Cellulose sulfate as such is known in the state of the art. The manufacture of cellulose sulfate is e.g. disclosed in the DD 299313. As disclosed in US 2003/181415 A sulfated polysaccharides, including cellulose sulfate, are known to be effective against various enveloped viruses and in particular herpes simplex virus (HSV), Papilloma viruses and HIV. The preparation disclosed therein is used to prevent sexually transmitted infections. It is believed that cellulose sulfate binds to the lipid membrane of encapsulated viruses and prevents fusion to and infection of host cells.

It has now been demonstrated herein that cellulose sulfate shows significant anti-inflammatory action over cellulose, which has no immuno-modulating effects. It was completely surprising that such an effect could be achieved through sulfatation of cellulose. E.g. in the US 2006/0040896 A a heparin (a sulfated carbohydrate polymer of the glycosaminoglycan family, obtainable from liver) derivative is disclosed, wherein the anti-inflammatory efficacy was increased by desulfation.

Giroud and Timsit (Thérapie 1973, 28: 889-905) mention an “anti-inflammatory” effect of cellulose sulfate in a mouse oedema model. This effect presents itself as a reduced inflammatory result of a challenge with subcutaneous carrageenan as irritant. Cellulose sulfate is applied previously to the challenge to reduce the following effects of the irritant. Carrageenan itself can also be used as such an “anti-inflammatory” agent by injection similar to cellulose sulfate to counter the oedema formation. It was not shown that cellulose sulfate can reduce the symptoms generally of inflammations.

Cellulose sulfate can reduce the amount of kininogen through the formation of bradykinin—which actually has some inflammatory properties (Barabe and Huberdeau, Res Commun Chem Pathol Pharmacol. 1989 63(2): 281-4 (abstract); Wang and Teng, Eur J Pharmacol 1989 161(1):9-18 (abstract)). Bradykinin is a potent endothelium-dependent vasodilator, causes contraction of non-vascular smooth muscle, increasing vascular permeability. The effects of carrageenan in the mouse oedema model is based on four factors: release of mast-cell amines, the generation of eicosanoids, the migration of polymorphonuclear leukocytes and the actions of kinins such as bradykinin (Cirino et al. PNAS USA 86: 3428-3432). By diminishing the kininogen reservoir through cellulose sulfate pretreatment the oedema formation can thus be reduced.

Although it is the view of the inventors that such an effect of cellulose sulfate against carrageenan induced oedemas cannot be generalized to the broad term of an anti-inflammatory effect since no inflammation is actually treated but only the kinin dependent effect of carrageenan reduced, to avoid confusion, the anti-inflammatory effect according to the present invention is defined by the reduction of pro-inflammatory cytokines (e.g., TNF-alpha, IL-6, IFN-gamma) and/or growth factors (e.g. IL-2), and/or the inhibition of T-cell activation after PMA (Phorbolester)+Ionomycin stimulation (Jurkat T-cells, and PBMCs) and/or the inhibition of TNF-alpha promoter transcription in LPS stimulated DC18 cells. In particular preferred (solely) kinin dependent inflammations like the carrageenan induced oedema are excluded from the definition. The composition is not for the treatment of inflammation in order to only reduce the kininogen reservoir. Preferably the inflammation to be treated is an established inflammation (acute or chronic inflammation), excluding a pretreatment (or prevention of an expected inflammation). In particular preferred the inflammation to be treated according to the present invention is a chronic inflammation.

Also included herein is the use of chitosan as anti-inflammatory polymer, preferably in combination with another polymer, especially negatively charged polymer. Chitosan is a linear polysaccharide composed of randomly distributed β-(1-4)-linked D-glucosamine (deacetylated unit) and N-acetyl-D-glucosamine (acetylated unit). Chitosan is produced commercially by deacetylation of chitin, which is the structural element in the exoskeleton of crustaceans (crabs, shrimp, etc.). There is a great diversity among chitosan products depending on the degree of deacetylation and origin of chitin. Most chitosan preparations are hardly soluble in neutral pH conditions. However, especially preferred is the use of water soluble chitosan. The production of water soluble chitosan is not trivial, but has already been demonstrated by Jakwang Co Ltd (KR 2001/027073, KR 2003/079444, US 2002/155175), which is a commercial supplier for water soluble chitosan. Water soluble chitosan is preferably completely soluble without the formation of colloids or gelatinous phases. Preferably the polymer is >90%, in particular preferred >99% soluble (of its initial weight). Specifically preferred chitosan preparations, but also the general polymer preparations, are purified or purifyable by passage through a 0.22 μm pore sterilisation filter.

Without being limited to a specific theory it is assumed that the anti-inflammatory polymer of the present invention, e.g. cellulose sulfate binds to (and antagonizes) specific receptors of immune cells and prevents TNF-alpha production or release. TNF-alpha acts as activator for T-cells which in turn produce IFN-gamma. This is of particular significance in patients suffering from sepsis, where the TNF-gamma production is continuously triggered by bacterial antigens, which eventually leads to the septic shock syndrome, multiple organ failure and death.

Therefore in a preferred embodiment of the present invention the polymer is purified, in particular sterilely purified, e.g. through filtration through a sterilisation filter in soluble form.

In a preferred embodiment the pharmaceutical composition is for the treatment of inflammation of the skin or mucosa. The pharmaceutical preparation comprising the polymer is in particular used for the (topical) treatment of the following conditions involving inflammation of the skin (dermal, transdermal) or mucosa, e.g. that can be associated with persistent bacterial infections of the skin. These conditions include but are not limited to psoriasis, atopic dermatitis, neurodermitis, bullos diseases, Folliculitis, Erysipelas—St. Anthony's Fire, Hidradenitis Suppurativa, Rocky Mountain Spotted Fever—RMSF, Cuts, Scrapes, and Stitches involving bacterial Infection, Cutaneous Anthrax, Botulism, Plague, Tularemia, Skin Abscesses, Carbuncles, Cat Scratch Disease, Cellulitsis (superficial bacterial infection of the skin), Erysipelas, Furuncles, Furunculosis, Hidradenitis Suppurativa, Folliculits, Impetigo, Staphylococcus aureus, Methicillin resistant Staph. Aureus, Staphylococcal scalded skin syndrome, Toxic shock syndrome, Streptococcus pyogenes, Necrotising fasciitis, Scarlet fever, Gonorrhoea, Meningococcal disease, Erysipelothrix insidiosa, Haemophilus ducreyi, Haemophilus (cause of cellulitis in young children), Klebsiella rhinoscleromatis, Pseudomonas aeruginosa, Calymmatobacterium granulomatis, Treponema species cause syphilis, yaws and pinta, Borrelia species cause Lyme disease, Mycobacterium species cause tuberculosis, leprosy and atypical mycobacterial infections. Other conditions sometimes caused by bacterial infection include, Kawasaki disease (mucocutaneous lymph node syndrome), Pseudofolliculitis barbae (shaving bumps), Sarcoidosis, Scalp folliculitis, uticaria, Conjunctivitis, sepsis (e.g. septic shock) and rheumatism.

In another preferred embodiment the pharmaceutical composition is for the treatment of complete or partial autoimmune etiology. In particular, the pharmaceutical composition is used for the treatment the following diseases with a complete or partial autoimmune etiology:
Coeliac disease is a disease characterised by chronic inflammation of the proximal portion of the small intestine caused by exposure to certain dietary gluten proteins;
Crohn's disease is a form of inflammatory bowel disease characterized by chronic inflammation of the intestinal tract. Major symptoms include abdominal pain and diarrhea;
Lupus erythematosus is a chronic (long-lasting) autoimmune disease wherein the immune system, for unknown reasons, becomes hyperactive and attacks normal tissue;
Pemphigus is an autoimmune disorder that causes blistering and raw sores on skin and mucous membranes;
Psoriasis is a skin disorder in which rapidly-multiplying skin cells produce itchy, scaly inflamed patches on the skin;
Reiter's syndrome is an autoimmune attack on various body systems in response to a bacterial infection and the body's confusion over the HLA-B27 marker;
Temporal arteritis (also known as “giant cell arteritis”) is an inflammation of blood vessels, most commonly the large and medium arteries of the head. Untreated, the disorder can lead to significant vision loss.

Preferably the pharmaceutical preparation is in form of a preparation for topical, dermal, transdermal or mucosal use, preferably skin lotions, cremes, sprays or gargle solutions. The polymer is especially suitable for topical application to treat skin or mucosal inflammation. But also systemic, e.g. parenteral or oral (also for specific mucosal treatment), is possible, especially for low molecular weight polymers. The present invention also provides the use of the pharmaceutical preparations.

Preferably, the polymer has a molecular weight ranging from about 15000 to 3,000,000 Da. Preferably, the molecular weight is greater than about 500,000, or for systemic administration lower than 500,000 Da.

Also preferred is that the preparation comprises pharmaceutical carriers or additives. The term “carrier” refers to a diluent, e.g. water, saline, excipient, or vehicle with which the composition can be administered. For a solid or fluid composition the carriers or additives in the pharmaceutical composition may comprise SiO₂, TiO₂, a binder, such as microcrystalline cellulose, polyvinylpyrrolidone (polyvidone or povidone), gum tragacanth, gelatine, starch, lactose or lactose monohydrate, alginic acid, maize starch and the like; a lubricant or surfactant, such as magnesium stearate, or sodium lauryl sulphate; a glidant, such as colloidal silicon dioxide; a sweetening agent, such as sucrose or saccharin. Preferably the preparation comprises buffers or pH adjusting agents, e.g. selected from citric acid, acetic acid, fumaric acid, hydrochloric acid, malic acid, nitric acid, phosphoric acid, propionic acid, sulfuric acid, tartaric acid, or combinations thereof. Cellulose sulfate in the form of a pharmaceutically acceptable salt, for example sodium cellulose sulfate may also be used. Other pharmaceutically acceptable salts include, among others, potassium, lithium and ammonium cellulose sulfate.

The degree of sulfation of cellulose sulfate is preferably above 12% and most preferably about 17-18%, or maximal sulfation. This degree is considered as a high sulfation.

The polymer is preferably a homopolymer, e.g. pure cellulose sulfate, or alternatively a derivative heteropolymer.

Furthermore, preferably the polymer is in amounts between 0.01% and 20%, preferably between 0.1% and 10%, most preferred between 0.5% and 5% of the preparation (%-values are given in w/w-%). In the pharmaceutical preparation also other anti-inflammatory polymers than cellulose sulfate can be present, most preferably water soluble chitosan. For these polymers the same preferred concentration ranges apply.

The administration of the preparation is not limited to administrations at the same time of inflammation but can also be used before or after an inflammation, e.g. for prophylactic treatment, i.e. a treatment before an expected inflammation to reduce the force of the inflammation.

In the preparation only one anti-inflammatory agent, e.g. cellulose sulfate, may be present. Nevertheless, in a more preferred embodiment the pharmaceutical preparation comprises at least two different anti-inflammatory components, preferably one component is chitosan. As has been shown herein (examples) chitosan, especially water soluble chitosan (Jakwang), is an exceptional anti-inflammatory polymer (alone or in combination), which can be used in a preparation of the present invention. One of the at least two components, preferably polymers as described herein can be applied locally to a subject. Afterwards the second polymer can be applied which preferably results in the formation of a (bio)film such as by usage of positively and negatively charged polymers, preferably carbohydrate polymers. The effective amounts for film formation can be easily determined by the skilled man in the art.

Preferably the polymer is chitosan in addition to a negatively charged polymer, preferably a carbohydrate. The combination of chitosan with a negatively charged polymer can result in a stable gel or film perfectly suitable for a topical treatment of inflammation. The negatively charged polymer is preferably a sulfated polymer, such as cellulose sulfate or carrageenan, preferably lambda carrageenan. Preferably 0.01% to 5%, more preferred 0.1% to 1%, most preferred 0.1% to 0.5% carrageenan are used with 0.01% to 10%, preferably 0.1% to 5%, most preferred 1% chitosan.

Also preferred, the polymer is cellulose sulfate in addition to a positively charged polymer, preferably chitosan. The combination of the negatively charged cellulose sulfate with a positively charges polymer, especially a carbohydrate such as chitosan yields stable films for topical, dermal, transdermal, cutaneous or mucosal treatment.

The present invention is further illustrated by the following Figures and examples.

### FIGURES

FIG. 1: LPS induced TNF-alpha production is inhibited by Cellulose sulphate and water soluble Chitosan but not other biopolymers.

The y-axis shows the amount of TNF-alpha in pg/ml that is produced after 18 hours of incubation with LPS and the corresponding biopolymer. The black bars correspond to a test substance concentration of 330 μg/ml. The numbers at the x-axis indicate the corresponding Biopolymers whereas 1 is Carboxymethylcellulose T70 (Niklacell, Mare GmbH, Austria), 2 is water soluble Chitosan (Jakwang, Korea), 3 Carboxymethylchitosan, 4 is Carrageenan (Carbamer), 5 is Carboxymethylcellulose T35 (Niklacell, Mare GmbH, Austria), 6 is kappa Carrageenan (Sigma), 7 is lambda Carrageenan (Sigma), 8 is cellulose sulfate (Acros), 9 uninduced not treated blood, 10 LPS induced blood and 11 is the Dexamethasone (Sigma) reference control 100 ng/ml.

FIG. 2: The inhibition of TNF-alpha production by Cellulose sulphate and Chitosan is dose dependent

The y-axis shows the amount of TNF-alpha in pg/ml produced after 18 hours of incubation with LPS and the corresponding biopolymer. The X-axis shows the test substance concentration in μg/ml at a logarithmic scale. Cellulose sulphate data is shown in diamonds and the corresponding fit as black line. Chitosan is shown as grey squares and the fit as dashed line.

FIG. 3: Cellulose Sulphate and Chitosan are active by administration hours after stimulation

The y-axis shows the amount of TNF-alpha in pg/ml produced after 18 hours of incubation with LPS and the corresponding biopolymer at different time points. The black bars indicate the addition of the test substance at the same time with LPS. For grey bar data the test substance was added 1 h after the stimulus, dashed bars 2 hours after stimulus and white bars 4 hours after stimulus. Lane number 1 on the x-axis corresponds to Cellulose sulphate and number 2 to chitosan both at a concentration of 20 μg/ml. The wave shaped bars in lanes 3, 4 and 5 indicate the assay controls which are indicated as with LPS stimulus without substance, LPS+Dexamethasone reference substance and unstimulated blood respectively.

FIG. 4: A: TNF-alpha release in CFTL-12 mast cells after Phorbolester/Ionomycin stimulation; cells were either left unstimulated (1), stimulated with PMA/Ionomycin (2) or pre-treated with chitosan (200 μg/ml; 3) or dexamethasone (Dex; 300 nM; 4) for 30 min. prior stimulation for 6 h. Bars represent mean of quadruplicate experiment, standard deviation is indicated.

B: TNF-alpha release in CFTL-12 mast cells after IgE/antigen stimulation; cells were either left unstimulated (1), stimulated with PMA/Ionomycin (2) or pre-treated with chitosan (200 μg/ml; 3) or dexamethasone (Dex; 300 nM; 4) for 30 min. prior stimulation for 6 h. Bars represent mean of quadruplicate experiment, standard deviation is indicated.

FIG. 5: Inhibition of T-cell activation as determined in a reporter gene assay using Jurkat T-cells; cells were either left unstimulated (1) or stimulated with phorbolester/ionomycin (2); in addition, cells were pre-treated with 100 nm cylcosporin A (3) or cellulose sulphate in different concentrations: 200 μg/ml (4), 66 μg/ml (5), 22 μg/ml (6), 7.4 μg/ml (7), and 2.5 μg/ml (8); cells were incubated with the substances for 1 h prior stimulation with Phorbolester (20 ng/ml) and ionomycin (1 μg/ml) for 18 h.

### EXAMPLES

A number of polymers were compared for their ability to inhibit the LPS induced stimulation of whole blood as determined by the release of TNF alpha. Cellulose sulphate and high molecular weight chitosan inhibited the LPS induced TNF-alpha production. In contrast, a series of other biopolymers did not have an effect. In addition, cellulose sulphate is effective when it is administrated even 2 hours after the stimulus.

### Example 1

### Materials and Methods

The inducing or inhibitory effects of cellulose sulfate and other biopolymers were investigated using primary blood cells in combination with cytokine ELISAs such as TNF-alpha. The cells were either left non-stimulated or they were induced with 200 ng/ml LPS (Lipopolysaccharide). The cells were stimulated for 18 h and TNF-alpha secretion was measured by using a commercial TNF-alpha ELISA (R&D systems). The results are given in the figures.

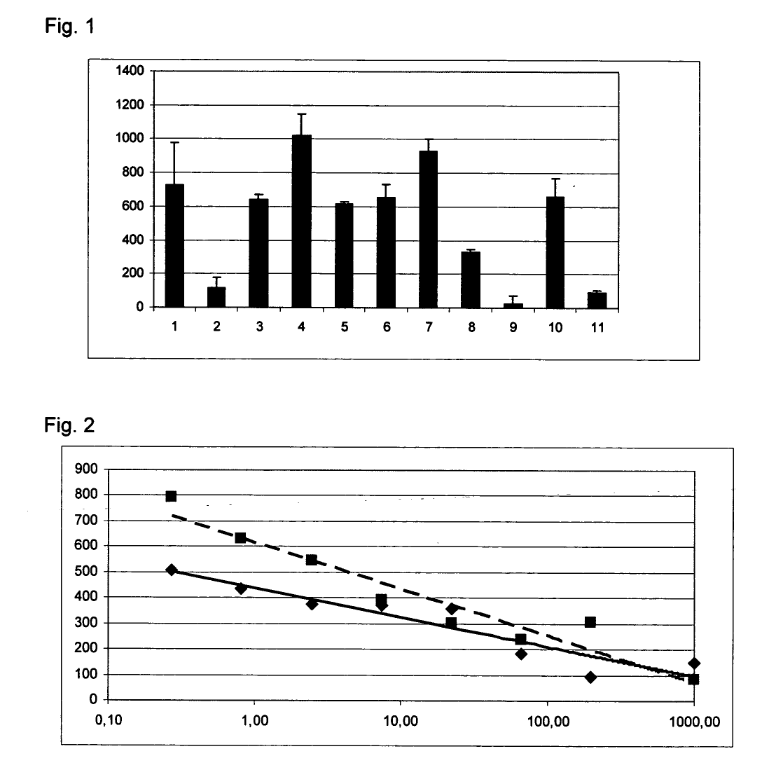

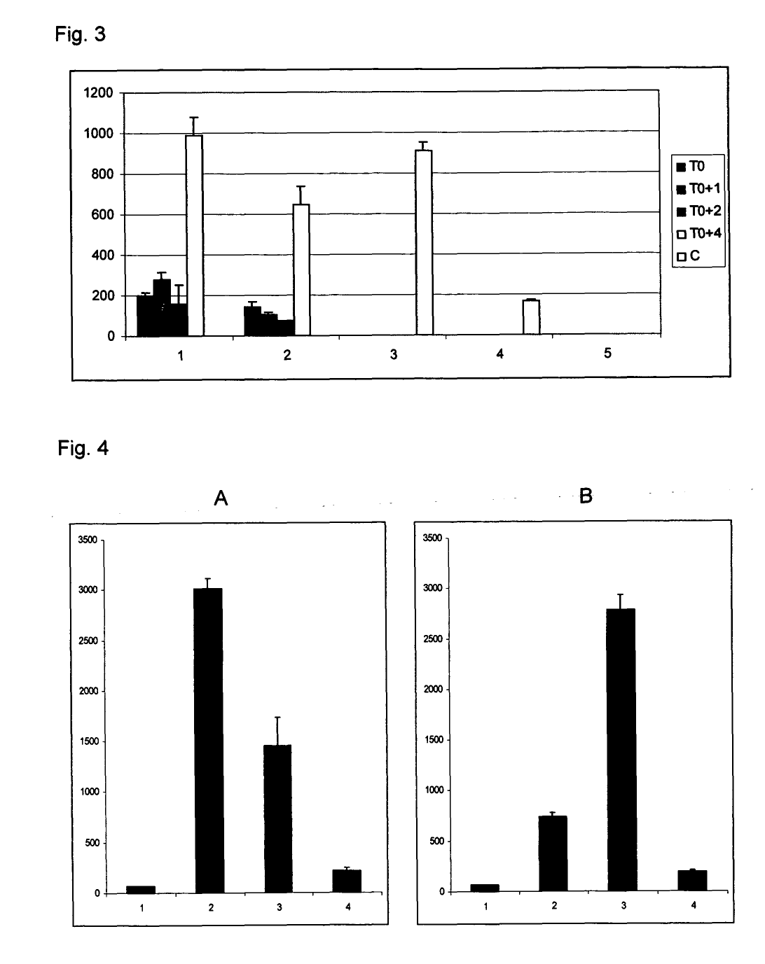

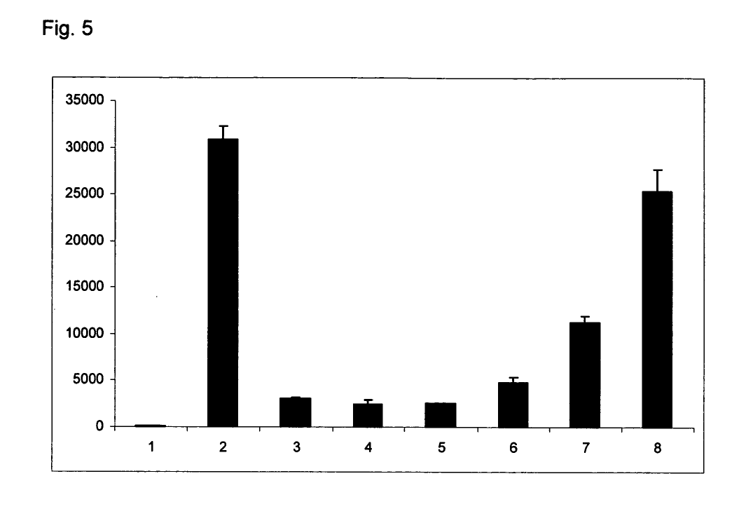

As it can be seen in FIG. 1 Chitosan (2) and cellulose sulphate (9) have a TNF-alpha inhibition activity. Other polymers do not have a significant inhibiting activity.

As shown in FIG. 2 the TNF-alpha inhibiting effect of Cellulose sulphate and Chitosan is dose dependent and the two polymers inhibit at comparable concentrations.

As shown in FIG. 3 Cellulose Sulphate and Chitosan are active up to 2 hours after stimulation. 2 hours after stimulation both polymers were as active as when added at the same time with LPS. When the polymers are added 4 hours after the stimulus the inhibitory effect is dramatically reduced.

### Example 2

### Activity of Cellulose Sulphate and Other Bioploymers on Immune Cells

Cellulose sulfate was investigated concerning its potential activating or inhibiting function on primary human blood cells. Several experiments that measure the induction of cytokines were performed that suggest that cellulose sulfate on its own is not immunostimulatory neither on blood cells nor on isolated lymphocytes. In contrast, cellulose sulfate markedly reduced the production of the pro-inflammatory cytokine TNF-alpha in LPS stimulated primary blood cells. Therefore, it is concluded that cellulose sulfate acts as a novel anti-inflammatory substance.

In an analogous experiment the immuno-stimulatory capacity of cellulose sulfate was determined. Cells were either left non-stimulated, activated with PMA+ionomycin or LPS or treated with 2 different batches of cellulose sulfate. Both batches did not lead to any induction of TNF-alpha release. In a similar setting also the cytokines IL-1 beta, IL-6, and IL-12 p40 were tested with identical results. Primary lymphocytes were tested in a similar setting for the capacity of cellulose sulfate to induce the release of IL-2 or IFN-gamma. Again no cytokine stimulation was detected.

Taken together the data indicate that cellulose sulfate does not provoke a cytokine release of primary blood cells or primary lymphocytes in-vitro, but exhibits a remarkable inhibitory effect on LPS stimulated primary blood cells.

Cellulose sulphate dose-dependently inhibits Jurkat T-cell activation with an IC50˜10 μg/ml. The results from the reporter gene assay were confirmed with an IL-2 ELISA. In analogy to Jurkat T-cells, PBMCs were stimulated with Phorbolester and Ionomycin in the absence or presence of cellulose sulphate (FIG. 5).

### Example 3

### Inhibition of TNF-alpha Production by Chitosan

Chitosan inhibits the TNF-alpha production in LPS and PMA stimulated whole blood cells (FIG. 4A). It also reduces the TNF-alpha production in PMA/Ionomycin stimulated CFTL-12 mast cells, but not in IgE/antigen stimulated mast cells. Chitosan reduces the TNF-alpha production of PMA/Ionomycin stimulated CFTL-12 cells but additionally enhances IgE/antigen dependent TNF-alpha release (FIG. 4B).

### Example 4

### Polymerisation of Chitosan with Carrageenan

The anti-inflammatory activity of chitosan suggests its topical application against diseases characterized by inflammatory processes e.g., psoriasis; especially disease stages complicated by secondary bacterial infections due to skin lesions can be treated by chitosan. In an attempt to generate a protective layer on the affected skin chitosan can be polymerized with sulphated polymers such as Carrageenan or cellulose sulphate resulting in a water insoluble but breathable membrane. Such a membrane protects the skin against further infections while reducing the already ongoing inflammation.

In order to test the potential polymerization partners of chitosan different types of Carrageenan were tested. While iota and kappa Carrageenan do not result in a stable polymerized gel at the tested concentrations lambda Carrageenan is the preferred polymerization partner for chitosan. As shown in table 1 between 0.1% and 1% Carrageenan can be used with 1% chitosan. However, preferred is a concentration between 0.5%-0.1%. Instead of lambda Carrageenan highly sulphated cellulose sulphate may be used.

TABLE 1
Polymerisation time of Chitosan (different concentrations see
top line) with different carrageenan (left side); the polymerisation
time was determined analogous to a coagulation assay.

| | 0.1% | 0.5% | 1% |
|---|---|---|---|
| | Chitosan | Chitosan | Chitosan |
| iota Carrageenan | | | |
| 0.10% | n.d. | n.d. | >40 s |
| 0.5% (gel at room temp.) | n.d. | n.d. | n.d. |
| 1% (gel at room temp.) | n.d. | n.d. | n.d. |
| kappa Carrageenan | | | |
| 0.10% | n.d. | n.d. | >40 s |
| 0.5% (gel at room temp.) | n.d. | n.d. | n.d. |
| 1% (gel at room temp.) | n.d. | n.d. | n.d. |
| lambda Carrageenan | | | |
| 0.10% | >40 s | 3 s | 2 s |
| 0.50% | 3 s | 3 s | 2 s |
| 1% | 2 s | 4 s | 4 s |

S means second, n.d., not determined.

1.-16. (canceled)

17. An anti-inflammatory pharmaceutical composition comprising a polymer defined as cellulose sulfate or chitosan with the proviso that, if the polymer is chitosan then the composition further comprises a negatively charged polymer.

18. The pharmaceutical composition of claim 17, wherein the preparation is in a form adapted for topical, dermal, transdermal, or mucosal use.

19. The pharmaceutical composition of claim 18, further defined as a skin lotion, cream, spray, or gargle solution.

20. The pharmaceutical composition of claim 17, further defined as comprising pharmaceutical carriers or additives.

21. The pharmaceutical composition of claim 17, further defined as sterile.

22. The pharmaceutical composition of claim 17, wherein the polymer is comprised in an amount of from 0.01% to 20% of the preparation.

23. The pharmaceutical composition of claim 22, wherein the polymer is comprised in an amount of from 0.1% to 10% of the preparation.

24. The pharmaceutical composition of claim 23, wherein the polymer is comprised in an amount of from 0.5% to 5% of the preparation.

25. The pharmaceutical composition of claim 17, further defined as comprising at least two different anti-inflammatory components.

26. The pharmaceutical composition of claim 25, wherein two of the at least two different anti-inflammatory components are chitosan and a negatively charged polymer.

27. The pharmaceutical composition of claim 26, wherein the negatively charged polymer is a sulfated polymer.

28. The pharmaceutical composition of claim 27, wherein the sulfated polymer is cellulose sulfate.

29. The pharmaceutical composition of claim 27, wherein the sulfated polymer is carrageenan.

30. The pharmaceutical composition of claim 29, wherein the sulfated polymer is lambda carrageenan.

31. The pharmaceutical composition of claim 24, wherein the polymer is cellulose sulfate and the composition further comprises a positively charged polymer.

32. The pharmaceutical composition of claim 31, wherein the positively charged polymer is chitosan.

33. A method of treating inflammation comprising:
obtaining a pharmaceutical composition of claim 17; and
administering the composition to a subject;
wherein inflammation in the subject is treated.

34. The method of claim 33, further defined as a method of treating inflammation of the skin or mucosa.

35. The method of claim 33, further defined as a method of treating psoriasis, atopic dermatitis, neurodermitis, bullos diseases, folliculitis, erysipelas, hidradenitis suppurativa, Rocky Mountain Spotted Fever, bacterial infected cuts, scrapes and stitches, cutaneous anthrax, botulism, plague, tularemia, skin abscesses, carbuncles, Cat Scratch Disease, cellulitsis, erysipelas, furuncles, furunculosis, hidradenitis suppurativa, folliculits, impetigo, staphylococcus aureus, staphylococcal scalded skin syndrome, toxic shock syndrome, streptococcus pyogenes, necrotising fasciitis, scarlet fever, gonorrhoea, meningococcal disease, erysipelothrix insidiosa, haemophilus ducreyi, haemophilus, klebsiella rhinoscleromatis, pseudomonas aeruginosa, calymmatobacterium granulomatis, treponema, borrelia, mycobacterium, leprosy, atypical mycobacterial infections, Kawasaki disease, pseudofolliculitis barbae, sarcoidosis, scalp folliculitis, uticaria, conjunctivitis, sepsis, and/or rheumatism.

36. The method of claim 35, further defined as a method of treating methicillin resistant staphylococcus aureus, syphilis, yaws, pinta, lyme disease, and/or tuberculosis.

37. The method of claim 33, further defined as a method of treating complete or partial autoimmune etiology.

38. The method of claim 33, further defined as a method of treating Coeliac disease, Crohn's disease, Lupus erythematosus, Pemphigus, Psoriasis, Reiter's syndrome and/or temporal arteritis (“giant cell arteritis”).

39. The method of claim 33, wherein the inflammation is an established inflammation.

40. The method of claim 39, wherein the established inflammation is acute or chronic inflammation.
